(19) United States
(12) Patent Application Publication
(10) Pub. No.: US 2011/0029011 A1
(43) Pub. Date: Feb. 3, 2011

(54) Clotting method for the repair of vascular defects and malformations

(76) Inventor: Ashish Awasthi, Cranbury, NJ (US)

Correspondence Address: Hollstein Keating Cattell Johnson & Goldstein P.C.;Willow Ridge Executive Office Park, Suite 301, 750 Rt. 73S., Marlton, NJ, 08053, US

(21) Appl. No.: 12/462,391

(22) Filed: Aug. 3, 2009

(51) Int. Cl. A61B 17/08; A61M 25/00

(52) U.S. Cl. 606/213; 604/264

(57) ABSTRACT

A method of sealing damaged and defective vessels utilizes stable thrombin and non-thrombin based solutions, gels, and biological glues to coat the tips of surgical appliances, such as surgical wires and trocars, with concentrations sufficient to cause clotting. The surgical wire or other appliance, with clotting material at its tip, is positioned within a protective sheath, such as a catheter or needle. The sheath provides a barrier which prevents clotting material to insinuate itself into healthy tissue. In the preferred embodiment, the protective catheter containing the wire is then inserted into the body of the patient and directed to the damaged site. The wire is then partially extended out of the sheath to allow the clotting material to be applied and deposited onto the damaged vessel, thereafter the wire and sheath are safely withdrawn from the body of the patient.

### BACKGROUND OF THE INVENTION

The method of the present invention addresses the sealing of damaged or defective tissue, whether congenital or acquired, e.g. vascular punctures, aneurysms, pseudoaneurysms, fistulas, ateriovenous malformations (AVM), and similar lumens. It is critical that such tissue be treated effectively and completely reduce and then stop blood flow from such tissues and initiate the body's own hemostatic processes.

Thrombin is an enzyme which reacts with soluble fibrinogen, a blood plasma protein, converting it to fibrin. Fibrin is the basis and is essential for clotting of blood. As a result, thrombin has found widespread usage in surgical closure and hemostatic methods. In addition, thrombin is used in many surgical glues for obtaining hemostatsis on surgical incisions or puncture locations. Such surgical glues are directly applied to the damaged or defective tissue or by application on the skin to stop blood flow following surgery.

Additionally, thrombin solutions have been used for treating post-procedure complications, including pseudoaneurysms, by direct injection into the false lumen of these defects to stop blood flow and coagulate the defect, with spontaneous resolution over time.

Other non-thrombin based procedures have been used to shrink atriovenous defects in high risk areas such as the brain, before treatment with other options. For example, these techniques employ a catheter with a balloon at one end to stop spillage of the material, as it is highly thrombogenic. Small aneurysms in difficult to reach areas of the brain have been treated by coils in order to cause the aneurysms to clot.

Successful as such techniques may be, they carry a significant risk of initiating clotting in normal tissue as a result of spillage of thrombin and other non-thrombin solutions. Additionally, thrombin injections are not used for intravascular procedures, due to the extreme and dangerous risk of clotting. Coils are, on occasion, difficult to deliver and thus unable to provide the required treatment.

### SUMMARY OF THE INVENTION

Thus, the method of the present invention is directed towards addressing the disadvantages and limitations of prior clotting material delivery techniques and thus improve treatment of damaged and defective vessels which must be sealed.

It is thus the object of the present invention to provide a method which allows for the effective and ready delivery of clotting material to defects within blood vessels and other tissues in a safe and efficient manner.

It is a further object of the present invention to provide a method for sealing damaged and defective vessels which provides for a barrier between blood/tissue and the clotting material until the material reaches the targeted defective site.

It is still another object of the present invention to provide a method for sealing damaged and defective vessels which allows for delivery of sufficient clotting material to be provided to the defective site.

It is another object of the present invention to provide a method for sealing damaged vessels such that the clotting material does not detach and embolize, thereby straying to an undesired location which would cause dangerous clotting and other serious problems.

These and other objects are accomplished by the present invention, a method of sealing damaged and defective vessels which utilizes stable thrombin and non-thrombin based solutions, gels, and biological glues to coat the tips of surgical appliances, such as surgical wires and trocars, with concentrations sufficient to cause clotting. The surgical wire or other appliance, with clotting material at its tip, is positioned within a protective sheath, such as a catheter or needle. The sheath provides a barrier which prevents clotting material to insinuate itself into healthy tissue. In the preferred embodiment the protective catheter containing the wire is then inserted into the body of the patient and directed to the damaged site. The wire is then partially extended out of the sheath to allow the clotting material to be applied and deposited at the site of damaged vessel, thereafter the wire and sheath are safely withdrawn from the body of the patient.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 shows the positioning of the wire used in the method of the present invention, within its protective sheath, i.e. a catheter.

FIG. 2 is a cross-sectional view of FIG. 1.

FIG. 3 is a view similar to FIG. 1, but showing the wire as it begins its travel through and out of the catheter.

FIG. 4 shows an alternate means of controlling the opening and closing of the catheter used in the method of the present invention.

FIG. 5 depicts a preliminary step of the method of the present invention.

FIG. 6 shows a subsequent step of the method of the present invention.

FIG. 7 shows a later step of the method of the present invention.

### DETAILED DESCRIPTION OF THE INVENTION

The unique method of the present invention utilizes a surgically protective sheath, such as a hollow catheter, as a barrier to prevent the clotting material to be applied and deposited on the damaged vessel/tissue, to come into contact with healthy, normal tissue, blood, and other susceptible areas unrelated to the treatment. An elongated appliance such as surgical wire or a trocar is configured to be positioned within and travel through the protective sheath to the damaged tissue to be clotted and closed.

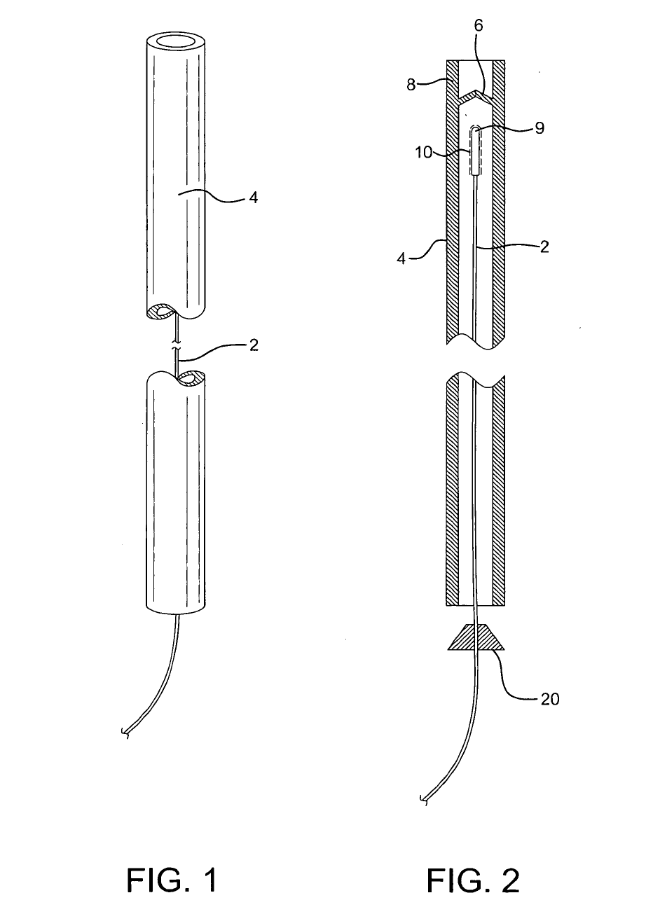

In the preferred embodiment, flexibly, maneuverable wire 2 in the range of 0.014 to 0.026 inches in diameter, is positioned to be advanced through catheter 4, ranging in diameter for 0.018 to 0.035 inches in diameter. (See FIG. 1). Clotting material 10, such as thrombin, biological glues or other sealant, is deposited on tip 9 of wire 2. It is contemplated that approximately 5-10 mm of the end of wire 2 will be coated with clotting material 10, as appropriate for the specific application.

Catheter 4 has a one way valve 6 at its distal end 8 which is normally closed, but which will permit wire 2, its tip 9 coated with clotting material 10, to pass through and out of the catheter. (See FIG. 2). Alternatively, catheter 4a can be opened and closed by a petal closure device. That is, it can have two wire strands 12 and 14 woven into its sides. Strands 12 and 14 are connected to biased, rotatable plates 16 and 18 which are normally closed, but which are configured to be opened when the strands are pulled. The opening and closing of distal end 8 of catheter 4 can also be accomplished by other recognized means. The herein invention is not to be considered restricted to the type of catheter closure device which is used.

After tip 9 of wire 2 is coated with clotting material 10, the wire is positioned in catheter 4. It is critical that wire 2 with its clotting material remain closeted within catheter 4 to avoid clotting material contact with healthy blood and tissue. Removeable backstop 20 is provided around wire 2 to prevent wire 2 from extending out of catheter 4 until such is needed.

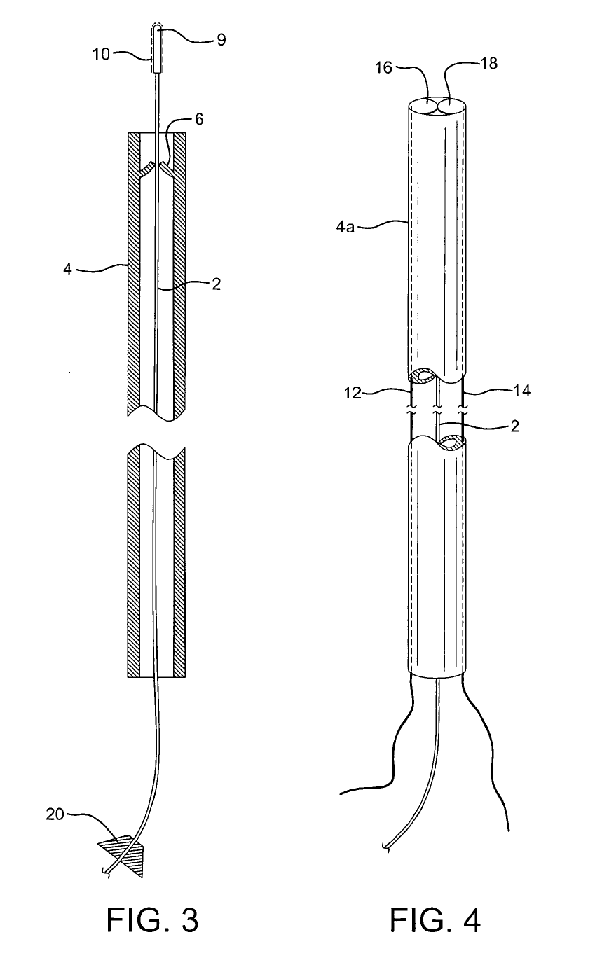

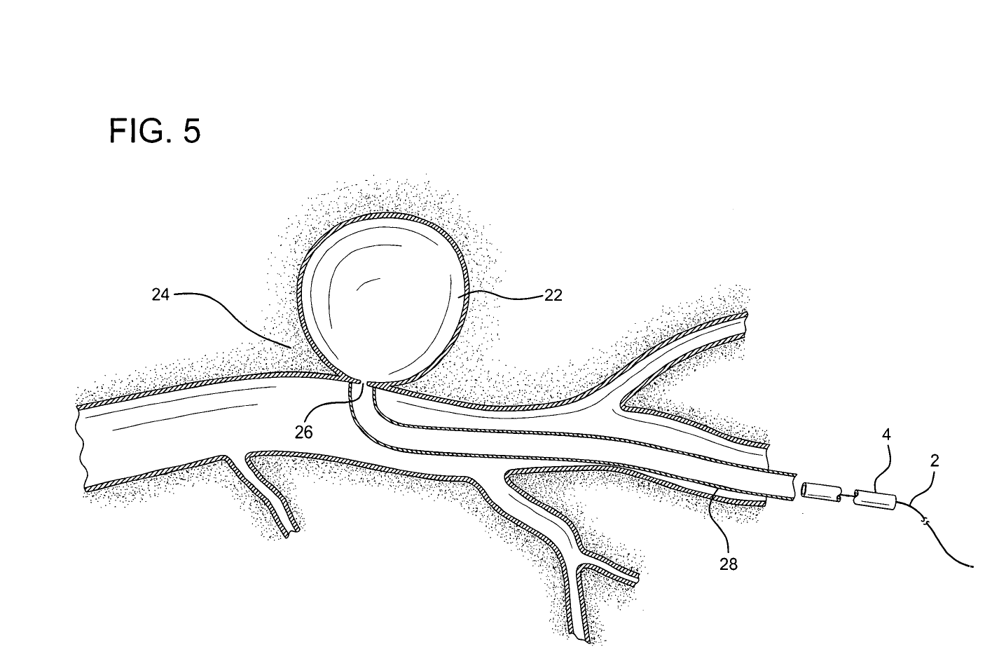

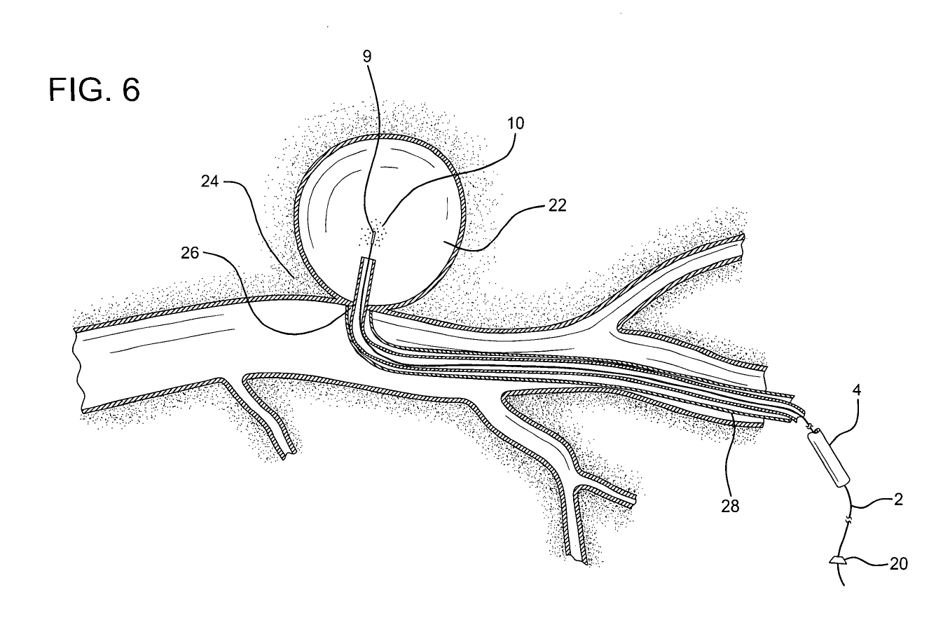

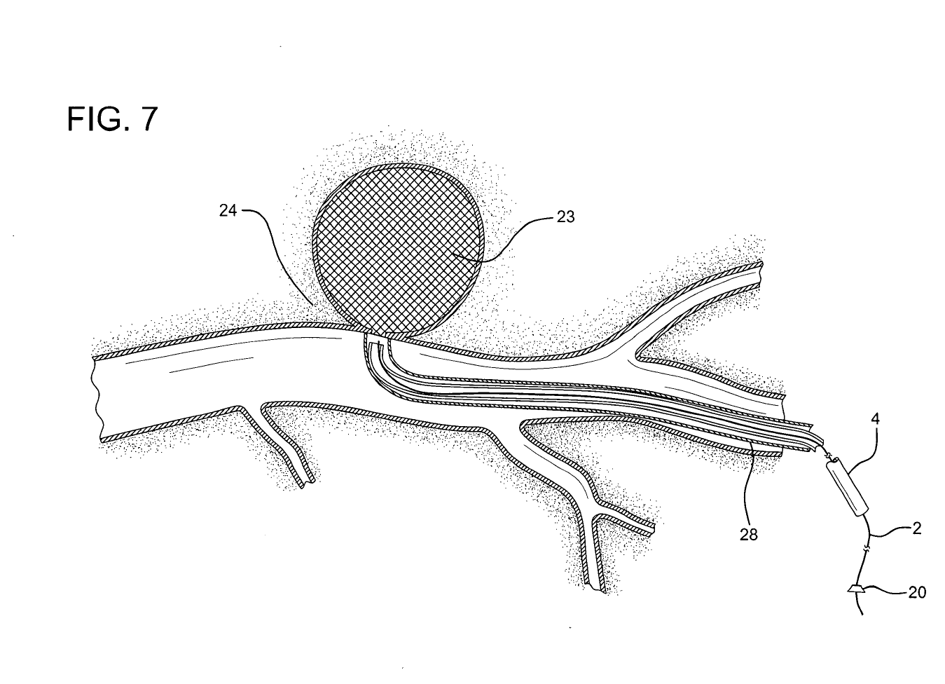

After the exact location of the damage or defective vessel, e.g. hematoma or aneurysm 22, is determined, catheter 4 containing coated wire 2 is inserted into body 24 of the patient, through preplaced catheter 28, and directed to the damaged tissue 22 and any puncture or lumen 26. (See FIG. 5). Backstop 20 is removed, distal end 8 of catheter 4 is opened, and wire 2, coated with clotting material 10, is pushed or otherwise compelled through and partially out of the catheter to repair or treat the damaged vessel 22. (See FIGS. 3 and 6). The damaged vessel, e.g. the aneurysm 23 in FIG. 7, is clotted. Catheter 4 and wire 2 are then removed together from the site. (See FIG. 7). The procedure is completed. Optionally, only wire 2 may be removed from the patient and another wire inserted into catheters 4 and 28 to repeat the procedure or for additional activities. The same catheters can be reused.

Alternatively, for superficial pseudoaneurysms such as occurs in the leg where thrombin is directly injected through a hollow gauge needle, a smaller inserter (e.g. a trocar) with its tip coated can be inserted into the clot. Treatment of the pseudoaneurysm avoids the risk of embolization and limb jeopardy which exists with injected thrombin.

Thus the current invention provides a unique method of applying thrombin, glue, or other biological clotting sealant to treat aneurysms, pseudoaneurysms, and similarly punctured or damaged vessels, while avoiding contact of the sealant with ambient tissue and blood. Targeted clotting occurs in a controlled manner. The method has application in the use of vascular seals in a variety of fields, including but not limited to cerebral, uterine, and coronary.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A method for sealing wounds in damaged or defective vessels such as aneurysms, pseudoaneurysms, punctured vascular tissue, ateriovenous malformations, fistulas, and other lumens within the body of a patient comprising the steps of:
providing a surgically protective elongated, hollow sheath;
providing clotting material for sealing the damaged vessel;
providing an elongated, maneuverable appliance for delivery of the clotting material;
applying the clotting material to the tip of the appliance;
positioning the appliance containing the clotting material in the sheath;
locating the damaged vessel to be sealed;
inserting the sheath containing the appliance into the body of the patient;
directing the sheath containing the appliance to the located damaged vessel;
compelling the appliance through and partially out of the sheath to the damaged vessel;
applying the clotting material located on the tip of the appliance directly to the damaged vessel to seal the damage to the vessel; and
withdrawing the sheath and appliance from the body of the patient.

2. The method as in claim 1 wherein the sheath is a catheter having a distal end which can be operably opened and closed.

3. The method as in claim 2 comprising the further step of closing the distal end of the catheter prior to its insertion into the body of the patient and while it is being directed to the damaged vessel.

4. The method as in claim 3 comprising the further step of opening the distal end of the catheter to allow the appliance to proceed out of the catheter.

5. The method as in claim 4 comprising the further step of closing the distal end of the catheter before withdrawing it and the appliance from the body of the patient.

6. The method as in claim 1 comprising the further step of restricting the movement of the appliance out of the catheter until the sheath containing the appliance is directed to the damaged vessel.

7. The method as in claim 6 comprising the further step of eliminating the restriction of movement of the appliance within the sheath to permit the appliance to extend out of the sheath.

8. The method as in claim 1 wherein the appliance is a flexible wire.

9. The method as in claim 1 wherein the appliance is a trocar.

10. The method as in claim 1 wherein the sheath is a hollow needle.

11. The method as in claim 1 wherein the clotting material is a sealant.

12. The method as in claim 1 wherein the clotting material is thrombin.
